(19) United States
(12) Patent Application Publication
(10) Pub. No.: US 2008/0214623 A1
(43) Pub. Date: Sep. 4, 2008

(54) N-(2,2-Dimethylpropyl)-6- -3-Pyridinecarboxamide

(76) Inventors: Amrik Chandi, Hertfordshire (GB); Trevor Raymond Keel, Hertfordshire (GB); Vipulkumar Kantibhai Patel, Hertfordshire (GB); Ann Louise Walker, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/917,534

(22) PCT Filed: Jun. 16, 2006
(86) PCT No.: PCT/GB2006/002212

§ 371 Date: Dec. 14, 2007

(30) Foreign Application Priority Data
Jun. 17, 2005 (GB) 0512429.2

(51) Int. Cl. A61K 31/4439; C07D 213/02; A61P 19/00; A61P 17/00; A61P 11/00; A61P 9/00; A61P 25/00; A61P 21/00; A61P 35/00; A61P 31/00; A61P 29/00

(52) U.S. Cl. 514/340; 546/272.1

(57) ABSTRACT

The present invention relates to a novel compound, processes for its preparation, compositions comprising the same and its use in the treatment of condition or diseases mediated by p38 kinase activity.

This invention relates to N-(2,2-dimethylpropyl)-6-{3-fluoro-5-[(3-isoxazolylamino)carbonyl]-2-methylphenyl}-3-pyridinecarboxamide, polymorphic forms thereof and its use as a pharmaceutical, particularly as a p38 kinase inhibitor, for the treatment of conditions or disease states mediated by p38 kinase activity or mediated by cytokines produced by the activity of p38 kinase.

WO 03/068747 describes nicotinamide derivatives that are inhibitors of p38 kinase. However, according to the present invention it has now been found that N-(2,2-dimethylpropyl)-6-{3-fluoro-5-[(3-isoxazolylamino)carbonyl]-2-methylphenyl}-3-pyridinecarboxamide, namely

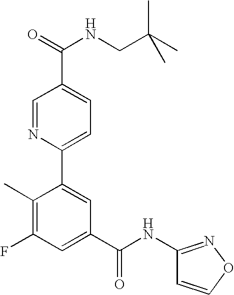

has potent p38 kinase inhibitory activity together with a pharmacokinetic profile which may make it particularly suitable for development as a pharmaceutical.

According to the invention there is provided N-(2,2-dimethylpropyl)-6-{3-fluoro-5-[(3-isoxazolylamino)carbonyl]-2-methylphenyl}-3-pyridinecarboxamide, or a pharmaceutically acceptable derivative thereof.

In one embodiment of the invention there is provided N-(2,2-dimethylpropyl)-6-{3-fluoro-5-[(3-isoxazolylamino)carbonyl]-2-methylphenyl}-3-pyridinecarboxamide.

As used herein, the term “pharmaceutically acceptable” means a compound which is suitable for pharmaceutical use. Salts and solvates of the compound of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of the compound of the invention and its pharmaceutically acceptable salts and solvates.

As used herein, the term “pharmaceutically acceptable derivative”, means any pharmaceutically acceptable salt, solvate, or prodrug, of the compound of the invention, which upon administration to the recipient is capable of providing (directly or indirectly) the compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. In one embodiment, the pharmaceutically acceptable derivatives are salts and solvates. In a further embodiment, the pharmaceutically acceptable derivatives are salts.

The compound of the present invention may be in the form of and/or may be administered as a pharmaceutically acceptable salt. For a review on suitable salts see Berge et al., J. Pharm. Sci., 1977, 66, 1-19.

Typically, a pharmaceutical acceptable salt may be readily prepared by using a desired acid. The salt may precipitate from solution and be collected by filtration or may be recovered by evaporation of the solvent.

Salts of the compound of the present invention may, for example, comprise acid addition salts resulting from reaction of an acid with a basic nitrogen atom present in the compound of the invention. Salts encompassed within the term “pharmaceutically acceptable salts” refer to non-toxic salts of the compound of this invention. Suitable addition salts are formed from acids which form non-toxic salts and examples are benzenesulfonate, bisulfate, camsylate, edisylate, estolate, esylate, glutamate, hydrobromide, hydrochloride, hydroiodide, isethionate, maleate, mesylate, napsylate, nitrate, oxalate, phosphate, sulfate, tosylate and trifluoroacetate.

Those skilled in the art of organic chemistry will appreciate that many organic compounds can form complexes with solvents in which they are reacted or from which they are precipitated or crystallized. These complexes are known as “solvates”. As used herein, the term “solvate” refers to a complex of variable stoichiometry formed by a solute (in this invention, N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof) and a solvent. Such solvents for the purpose of the invention may not interfere with the biological activity of the solute. Examples of suitable solvents include water, methanol, chloroform, ethanol and acetic acid. In one embodiment, the solvent used is a pharmaceutically acceptable solvent. Examples of suitable pharmaceutically acceptable solvents include water, ethanol and acetic acid. In one embodiment, the solvent used is water. A complex with water is known as a “hydrate”. Solvates of the compound of the invention are within the scope of the invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series; Edward B. Roche, ed., Bioreversible Carriers in Drug Design, American Pharmaceutical Association and Pergamon Press, 1987; and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release N-(2,2-dimethylpropyl)-6-{3-fluoro-5-[(3-isoxazolylamino)carbonyl]-2-methylphenyl}-3-pyridinecarboxamide in vivo when such prodrug is administered to a patient. Prodrugs are generally prepared by modifying functional groups in a way such that the modification is cleaved, either by routine manipulation or in vivo, yielding the parent compound.

All polymorphic forms of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof are within the scope of the invention.

It will be appreciated that differing polymorphic forms of a pharmaceutically active chemical substance may differ from one another in terms of their properties such as their stability, solubility, dissolution rate and ultimately bioavailability. It will be further appreciated that polymorphic forms of a pharmaceutically active chemical substance may be characterised and differentiated using a number of conventional analytical techniques.

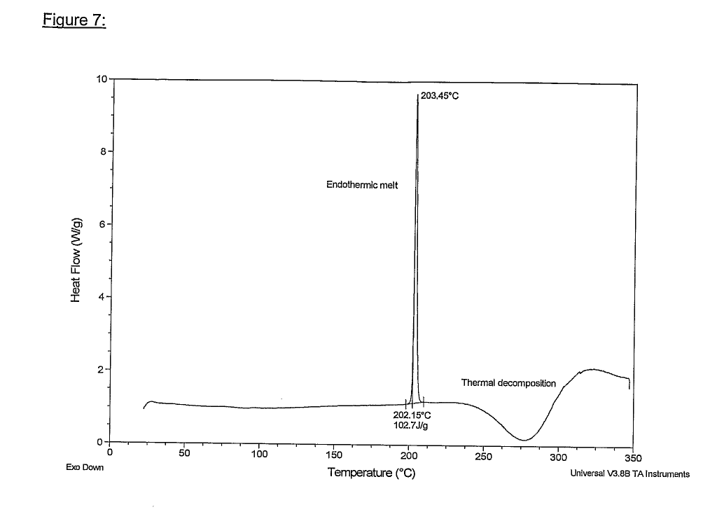

In a further aspect, the present invention provides a crystalline form of N-(2,2-dimethylpropyl)-6-{3-fluoro-5-[(3-isoxazolylamino)carbonyl]-2-methylphenyl}-3-pyridinecarboxamide (FORM 1) characterised by substantially the same X-ray powder diffraction (XRPD) pattern as shown in FIG. 1, wherein the XRPD pattern is expressed in terms of 2 theta angles and obtained with a diffractometer using copper Kα-radiation using procedures described herein and/or substantially the same differential scanning calorimetry (DSC) thermograms as shown in FIG. 6 wherein the DSC was performed at a scan rate of 10° per minute using a loosely covered aluminium pan using procedures described herein. The XRPD of Form 1 shows characteristic 2 theta angle peaks at 3.0±0.1, 6.0±01 and 13.5±0.1. The melting point of Form 1 is 187° C.±2° C.

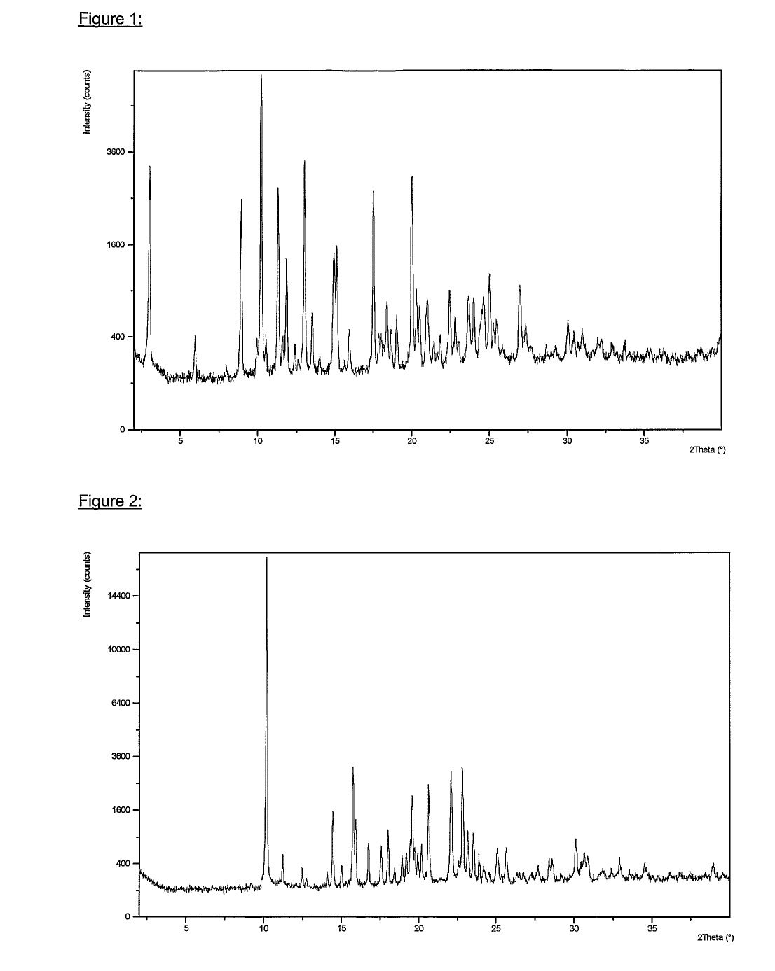

In a further aspect, the present invention provides a crystalline form of N-(2,2-dimethylpropyl)-6-{3-fluoro-5-[(3-isoxazolylamino)carbonyl]-2-methylphenyl}-3-pyridinecarboxamide (FORM 2) characterised by substantially the same X-ray powder diffraction (XRPD) pattern as shown in FIG. 2, wherein the XRPD pattern is expressed in terms of 2 theta angles and obtained with a diffractometer using copper Kα-radiation using procedures described herein and/or substantially the same differential scanning calorimetry (DSC) thermograms as shown in FIG. 7 wherein the DSC was performed at a scan rate of 10° per minute using a loosely covered aluminium pan using procedures described herein. The XRPD of Form 2 shows characteristic 2 theta angle peaks at 16.8±0.1 and 25.6±0.1. The melting point of Form 2 is 203° C.±2° C.

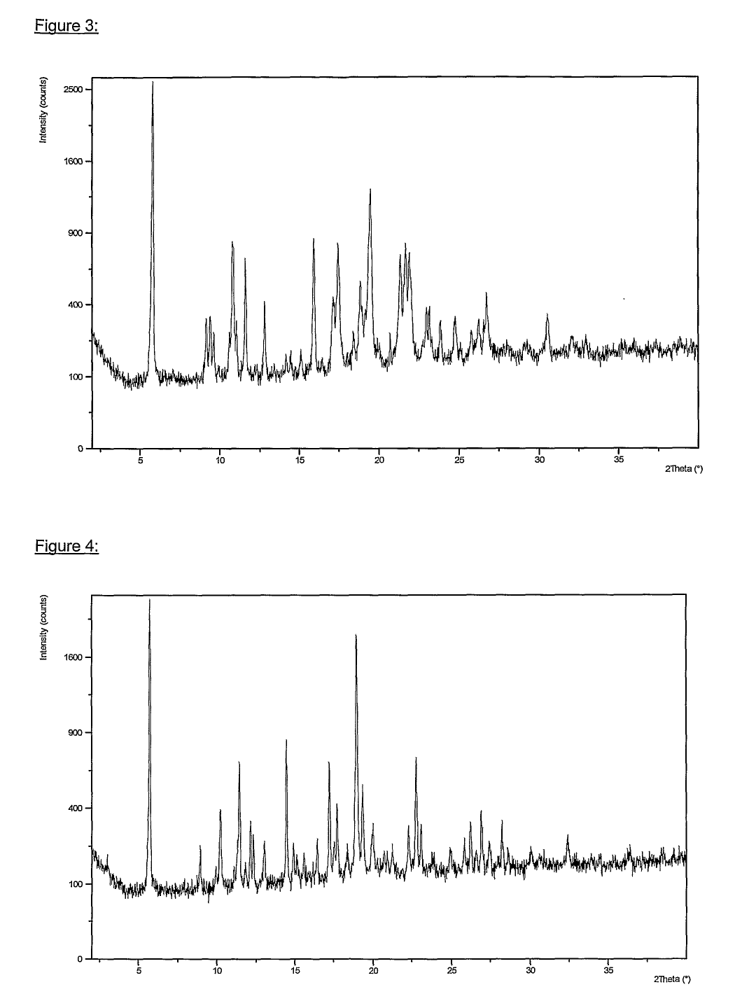

In a further aspect, the present invention provides a crystalline form of N-(2,2-dimethylpropyl)-6-{3-fluoro-5-[(3-isoxazolylamino)carbonyl]-2-methylphenyl}-3-pyridinecarboxamide (FORM 3) characterised by substantially the same X-ray powder diffraction (XRPD) pattern as shown in FIG. 3, wherein the XRPD pattern is expressed in terms of 2 theta angles and obtained with a diffractometer using copper Kα-radiation using procedures described herein. The XRPD of Form 3 shows characteristic 2 theta angle peaks at 9.2±0.1, 9.4 10.1 and 10.8±0.1.

In a further aspect, the present invention provides a crystalline form of N-(2,2-dimethylpropyl)-6-{(3-fluoro-5-[(3-isoxazolylamino)carbonyl]-2-methylphenyl}-3-pyridinecarboxamide (FORM 4) characterised by substantially the same X-ray powder diffraction (XRPD) pattern as shown in FIG. 4, wherein the XRPD pattern is expressed in terms of 2 theta angles and obtained with a diffractometer using copper Kα-radiation using procedures described herein. The XRPD of Form 4 shows characteristic 2 theta angle peaks at 12.2±0.1, 12.3±0.1 and 28.2±0.1.

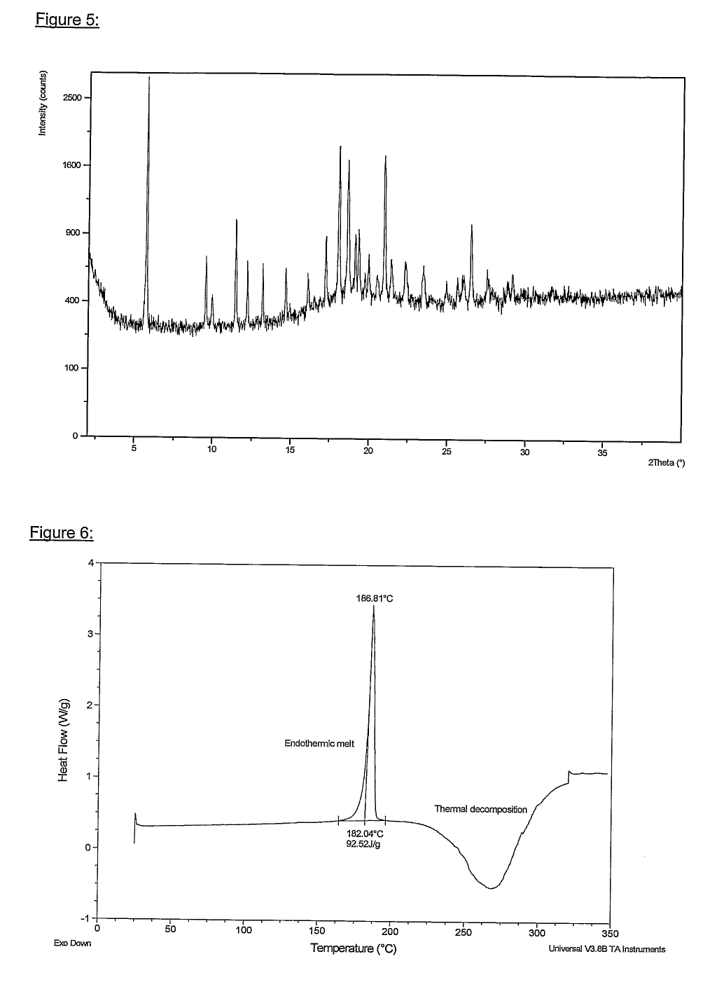

In a further aspect, the present invention provides a crystalline form of N-(2,2-dimethylpropyl)-6-{3-fluoro-5-[(3-isoxazolylamino)carbonyl]-2-methylphenyl}-3-pyridinecarboxamide (FORM 5) characterised by substantially the same X-ray powder diffraction (XRPD) pattern as shown in FIG. 5, wherein the XRPD pattern is expressed in terms of 2 theta angles and obtained with a diffractometer using copper Kα-radiation using procedures described herein. The XRPD of Form 5 shows characteristic 2 theta angle peaks at 16.1±0.1 and 26.5±0.1.

Forms 1-5 of N-(2,2-dimethylpropyl)-6-{3-fluoro-5-[(3-isoxazolylamino)carbonyl]-2-methylphenyl}-3-pyridinecarboxamide can be prepared according to procedures described herein.

The compound of this invention may be made by a variety of methods, including standard chemistry. Illustrative general synthetic methods are set out below and then the specific compound of the invention is prepared in the working Example.

N-(2,2-Dimethylpropyl)-6-{3-fluoro-5-[(3-isoxazolylamino)carbonyl]-2-methylphenyl}-3-pyridinecarboxamide may be prepared by reacting a compound of formula (I)

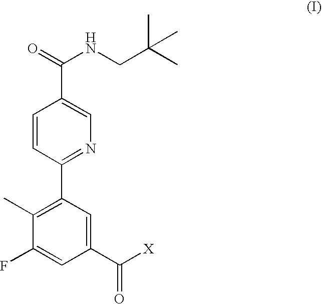

in which X is a leaving group, for example chlorine,
with an amine of formula (II)

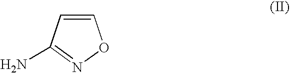

under suitable amide forming conditions.

Suitable amide forming conditions are well known in the art and include, for example, reaction of a compound of formula (I) with an amine of formula (II) in the presence of a base such as triethylamine and a solvent such as DCM.

A compound of formula (I) may be prepared by reacting a compound of formula (III)

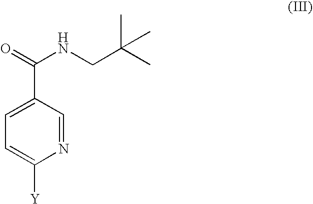

in which Y is halogen, for example chlorine,
with a compound of formula (IVA) or (IVB)

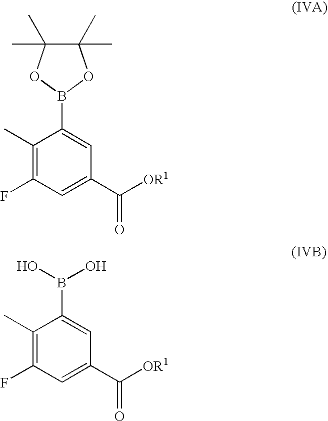

in which R¹ is a protecting group, for example C₁₋₆alkyl such as methyl,
in the presence of a catalyst, for example tetrakis(triphenylphosphine)palladium, and converting the group OR¹ to a leaving group.

A compound of formula (III) may readily be prepared by reacting a compound of formula (III)

in which Y is as hereinbefore defined,

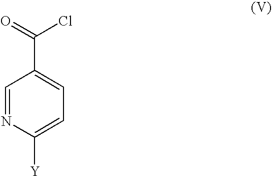

with an amine compound of formula (VI)

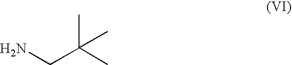

under amide forming conditions, for example, treating a solution of the compound of formula (V) in for example dichloromethane, with an amine of formula (VI) in the presence of potassium or sodium carbonate.

A compound of formula (IVA) may be prepared by reacting a compound of formula (VII)

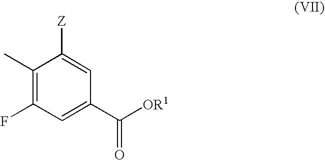

in which Z is halogen, for example iodine,
with bis(pinnacolato)diboron, Pd(dppf)Cl₂ and potassium acetate in a solvent such as DMF.

A compound of formula (IVB) may be prepared by, for example, reacting a compound of formula (VII) as hereinbefore defined with isopropylmagnesium chloride and triisopropylborate in a solvent such as THF.

Alternatively, N-(2,2-dimethylpropyl)-6-{3-fluoro-5-[(3-isoxazolylamino)carbonyl]-2-methylphenyl}-3-pyridinecarboxamide may be prepared by reacting a compound of formula (III) as hereinbefore defined,

with a compound of formula (VIIIA) or (VIIIB)

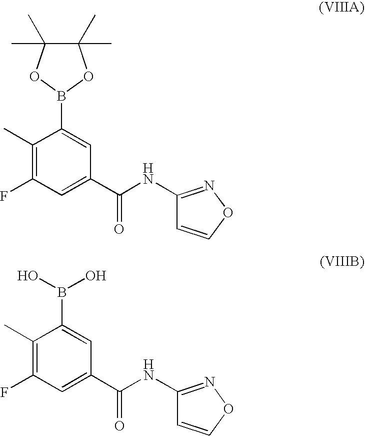

in the presence of a catalyst, for example tetrakis(triphenylphosphine)palladium.

The compounds of formula (VIIIA) and (VIIIB) may be prepared in an analogous manner to the compounds of formula (IVA) and (IVB).

For example, one method for preparing N-(2,2-dimethylpropyl)-6-{3-fluoro-5-[(3-isoxazolylamino)carbonyl]-2-methylphenyl}-3-pyridinecarboxamide comprises the reactions set out in Scheme 1 below.

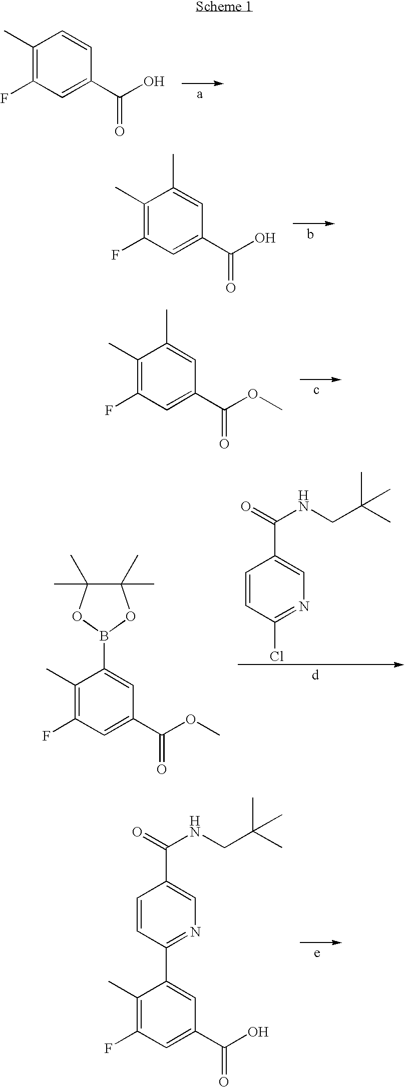

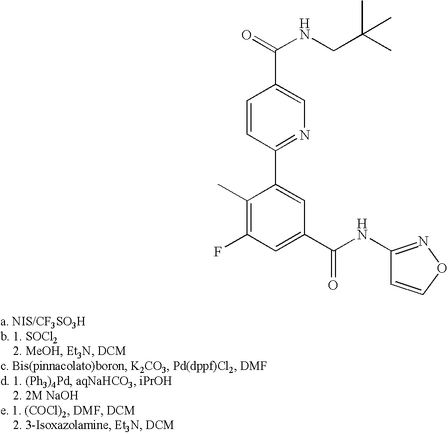

For example, a further method for preparing N-(2,2-dimethylpropyl)-6-{3-fluoro-5-[(3-isoxazolylamino)carbonyl]-2-methylphenyl}-3-pyridinecarboxamide comprises the reactions set out in Scheme 2 below.

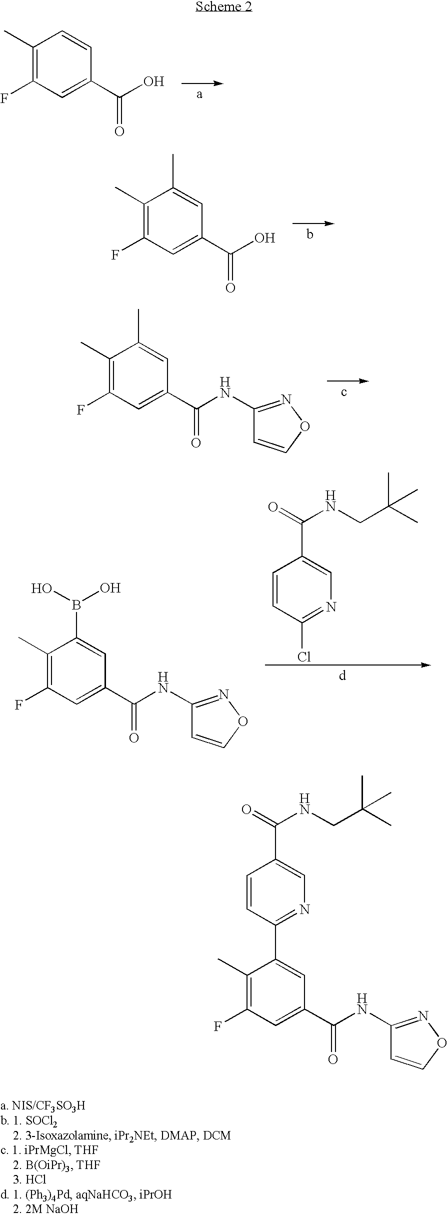

A yet further method for preparing N-(2,2-dimethylpropyl)-6-{3-fluoro-5-[(3-isoxazolylamino)carbonyl]-2-methylphenyl}-3-pyridinecarboxamide comprises the reactions set out in Scheme 3 below.

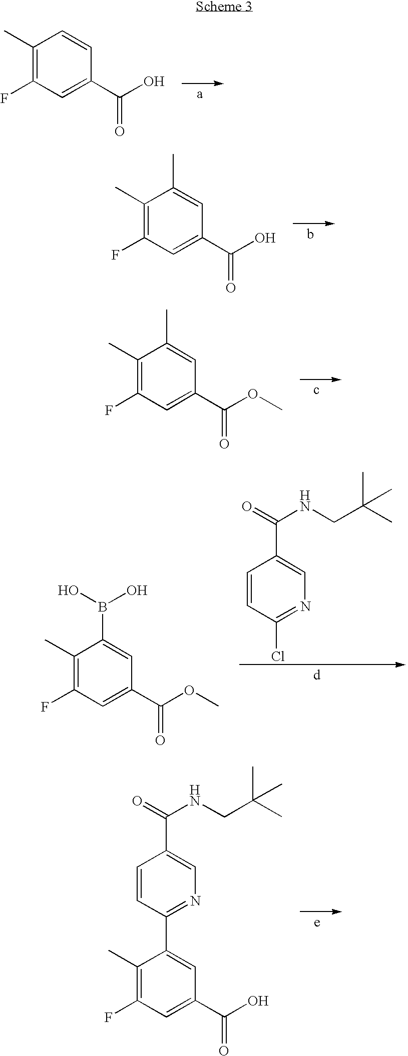

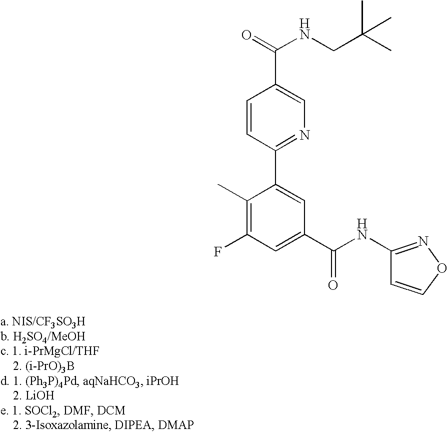

Whilst it is possible for the compound of the present invention to be administered as the raw chemical, the compound and its pharmaceutically acceptable derivatives are conveniently administered in the form of pharmaceutical compositions e.g. when the agent is in admixture with a suitable pharmaceutical excipient, diluent and/or carrier selected with regard to the intended route of administration and standard pharmaceutical practice.

Thus, in another aspect of the invention, we provide a pharmaceutical composition comprising N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof, in association with one or more pharmaceutically acceptable excipients, diluents and/or carriers. The excipient, diluent or carrier must be “acceptable” in the sense of being compatible with the other ingredients of the formulation and not deleterious to the recipient thereof.

According to a further aspect, the invention provides a pharmaceutical composition comprising, as active ingredient, N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof, in association one or more pharmaceutically acceptable excipients, diluents and/or carriers for use in therapy, and in particular in the treatment of human or animal subjects suffering from a condition susceptible to amelioration by an inhibitor of p38 kinase.

The present invention also provides a pharmaceutical composition comprising a therapeutically effective amount of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof, and a pharmaceutically acceptable excipient, diluent and/or carrier (including combinations thereof).

There is further provided by the present invention a process of preparing a pharmaceutical composition, which process comprises mixing N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof, together with a pharmaceutically acceptable excipient, diluent and/or carrier.

The pharmaceutical compositions may be for human or animal usage in human and veterinary medicine and will typically comprise any one or more of a pharmaceutically acceptable excipient, diluent or carrier. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical excipient, diluent or carrier can be selected with regard to the intended route of administration and standard pharmaceutical practice. The pharmaceutical compositions may comprise as—or in addition to—the excipient, diluent or carrier any suitable binder(s), lubricant(s), suspending agent(s), coating agent(s) and solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

For some embodiments, the agent of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO 91/11172, WO 94/02518 and WO 98/55148.

The compound of the invention may be milled using known milling procedures such as wet milling to obtain a particle size appropriate for tablet formation and for other formulation types. Finely divided (nanoparticulate) preparations of the compounds of the invention may be prepared by processes known in the art, for example see WO 02/00196 (SmithKline Beecham).

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The routes for administration (delivery) include, but are not limited to, one or more of: oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. It is to be understood that if the composition comprises more than one active component, then those components may be administered by different routes.

The compound of the invention and its pharmaceutically acceptable salts and solvates may be formulated for administration in any suitable manner. They may, for example, be formulated for topical administration or administration by inhalation, or for oral, transdermal or parenteral administration. The pharmaceutical composition may be in a form such that it can effect controlled release of the compound of the invention and its pharmaceutically acceptable derivatives. In one embodiment, the agents of the present invention are delivered systemically such as orally, buccally or sublingually. In a further embodiment, the method of administration, and corresponding formulation, is oral administration.

For oral administration, the pharmaceutical composition may take the form of, and be administered as, for example, tablets (including sub-lingual tablets) and capsules (each including timed release and sustained release formulations), ovules, pills, powders, granules, elixirs, tinctures, emulsions, solutions, syrups or suspensions prepared by conventional means with acceptable excipients for immediate-, delayed-, modified-, sustained-, pulsed- or controlled-release applications.

For instance, for oral administration in the form of a tablet or capsule, the active drug component can be combined with an oral, non-toxic pharmaceutically acceptable inert carrier such as ethanol, glycerol, water and the like. The tablets may also contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Examples of excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules can be made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additives such as peppermint oil or saccharin, and the like can also be added.

Where appropriate, dosage unit formulations for oral administration can be microencapsulated. The formulation can also be prepared to prolong or sustain the release as for example by coating or embedding particulate material in polymers, wax or the like.

The compound of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compound of the present invention can also be administered in the form of liposome emulsion delivery systems, such as small unilamellar vesicles, large unilamellar vesicles and multilamellar vesicles. Liposomes can be formed from a variety of phospholipids, such as cholesterol, stearylamine or phosphatidylcholines.

The compound of the present invention may also be delivered by the use of monoclonal antibodies as individual carriers to which the compound molecules are coupled. The compound of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compound of the present invention may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

The present invention includes pharmaceutical compositions containing 0.1 to 99.5%, for example, 0.5 to 90% of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof in combination with a pharmaceutically acceptable carrier.

Likewise, the composition may also be administered in nasal, ophthalmic, otic, rectal, topical, intravenous (both bolus and infusion), intraperitoneal, intraarticular, subcutaneous or intramuscular, inhalation or insufflation form, all using forms well known to those of ordinary skill in the pharmaceutical arts.

For transdermal administration, the pharmaceutical composition may be given in the form of a transdermal patch, such as a transdermal iontophoretic patch.

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent; and/or by using infusion techniques. For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilizing and/or dispersing agents. For administration by injection these may take the form of a unit dose presentation or as a multidose presentation optionally with an added preservative. Alternatively for parenteral administration the active ingredient may be in powder form for reconstitution with a suitable vehicle. For parenteral administration, the compound is best used in the form of a sterile aqueous solution which may contain other substances, for example, enough salts or glucose to make the solution isotonic with blood. The aqueous solutions should be suitably buffered (for example to a pH of from 3 to 9), if necessary. The preparation of suitable parenteral formulations under sterile conditions is readily accomplished by standard pharmaceutical techniques well-known to those skilled in the art.

The compositions of the present invention may be administered by direct injection.

The compound of the invention may also be formulated as a depot preparation. Such long acting formulations may be administered by implantation (for example subcutaneously or intramuscularly) or by intramuscular injection. Thus, for example, the compound of the invention may be formulated with suitable polymeric or hydrophobic materials (for example as an emulsion in an acceptable oil) or ion exchange resins, or as sparingly soluble derivatives, for example, as a sparingly soluble salt.

Alternatively the composition may be formulated for topical application, for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water.

Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

For administration by inhalation the compound according to the invention is conveniently delivered in the form of an aerosol spray presentation from pressurized packs or a nebulizer, with the use of a suitable propellant, e.g. dichlorodifluoromethane, trichlorofluoromethane, dichlorotetrafluoroethane, a hydrofluoroalkane such as tetrafluoroethane or heptafluoropropane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of a compound of the invention and a suitable powder base such as lactose or starch.

Alternatively, the compound of the present invention can be administered in the form of a suppository or pessary, or it may be applied topically in the form of a gel, hydrogel, lotion, solution, cream, ointment or dusting powder.

The compound of the present invention may also be administered by the pulmonary or rectal routes. It may also be administered by the ocular route. For ophthalmic use, the compound can be formulated as a micronised suspension in isotonic, pH adjusted, sterile saline, or as a solution in isotonic, pH adjusted, sterile saline, optionally in combination with a preservative such as a benzylalkonium chloride. Alternatively, it may be formulated in an ointment such as petrolatum.

The pharmaceutical compositions generally are administered in an amount effective for treatment or prophylaxis of a specific condition or conditions. Initial dosing in humans is accompanied by clinical monitoring of symptoms, such symptoms for the selected condition. In general, the compositions are administered in an amount of active agent of at least about 100 μg/kg body weight. In most cases they will be administered in one or more doses in an amount not in excess of about 20 mg/kg body weight per day. For example, in most cases, dose may be from about 100 μg/kg to about 5 mg/kg body weight, daily. For administration particularly to mammals, and particularly humans, it is expected that the daily dosage level of the active agent will be from 0.1 mg/kg to 10 mg/kg and typically around 1 mg/kg. It will be appreciated that optimum dosage will be determined by standard methods for each treatment modality and indication, taking into account the indication, its severity, route of administration, complicating conditions and the like. The physician in any event will determine the actual dosage which will be most suitable for an individual and will vary with the activity of the specific compound to be employed, the metabolic stability and length of action of that compound, age, weight, general health, sex, diet, mode and time of administration, rate of excretion, drug combination, severity of the particular condition and response of the particular individual. The effectiveness of a selected actual dose can readily be determined, for example, by measuring clinical symptoms or standard anti-inflammatory indicia after administration of the selected dose. The above dosages are exemplary of the average case. There can, of course, be individual instances where higher or lower dosage ranges are merited, and such are within the scope of this invention. For conditions or disease states as are treated by the present invention, maintaining consistent daily levels in a subject over an extended period of time, e.g., in a maintenance regime, can be particularly beneficial. For oral and parenteral administration to humans, the daily dosage level of the agent may be in single or divided doses.

In another aspect, the present invention provides N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof, for use in therapy.

The compound of the present invention is an inhibitor of the serine/threonine kinase p38 and is therefore also an inhibitor of cytokine production which is mediated by p38 kinase. Within the meaning of the term “inhibitors of the serine/threonine kinase p38” are included those compounds that interfere with the ability of p38 to transfer a phosphate group from ATP to a protein substrate according to the assay described below.

It will be appreciated that the compound of the invention may be selective for one or more of the isoforms of p38, for example p38α, p38β, p38γ and/or p38δ. In one embodiment, the compound of the invention selectively inhibits the p38α and p38β, isoforms. Assays for determining the selectivity of compounds for the p38 isoforms are described in, for example, WO 99/61426, WO 00/71535 and WO 02/46158.

It is known that p38 kinase activity can be elevated (locally or throughout the body), p38 kinase can be incorrectly temporally active or expressed, p38 kinase can be expressed or active in an inappropriate location, p38 kinase can be constitutively expressed, or p38 kinase expression can be erratic; similarly, cytokine production mediated by p38 kinase activity can be occurring at inappropriate times, inappropriate locations, or it can occur at detrimentally high levels.

Accordingly, the present invention provides a method for the treatment of a condition or disease state mediated by p38 kinase activity, or mediated by cytokines produced by the activity of p38 kinase, comprising administering to a subject in need thereof a therapeutically effective amount of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof. The compound may be administered as a single or mixture of polymorphic crystalline form or forms, or an amorphous form.

The present invention also provides a method of inhibiting cytokine production which is mediated by p38 kinase activity in a subject, e.g. a human, which comprises administering to said subject in need of cytokine production inhibition a therapeutic, or cytokine-inhibiting, amount of the compound of the present invention. The compound may be administered as a single or polymorphic crystalline form or forms, or an amorphous form.

The present invention treats these conditions by providing a therapeutically effective amount of the compound of this invention. By “therapeutically effective amount” is meant a symptom-alleviating or symptom-reducing amount, a cytokine-reducing amount, a cytokine-inhibiting amount, a kinase-regulating amount and/or a kinase-inhibiting amount of a compound. Such amounts can be readily determined by standard methods, such as by measuring cytokine levels or observing alleviation of clinical symptoms. For example, the clinician can monitor accepted measurement scores for anti-inflammatory treatments. It will be appreciated that reference to treatment includes acute treatment or prophylaxis as well as the alleviation of established symptoms.

The compound of the present invention can be administered to any subject in need of inhibition or regulation of p38 kinase or in need of inhibition or regulation of p38 mediated cytokine production. In particular, the compound may be administered to mammals. Such mammals can include, for example, horses, cows, sheep, pigs, mice, dogs, cats, primates such as chimpanzees, gorillas, rhesus monkeys, and humans. In one embodiment, the mammal is a human.

Thus, the present invention provides methods of treating or reducing symptoms in a human or animal subject suffering from, for example, rheumatoid arthritis, osteoarthritis, asthma, psoriasis, eczema, allergic rhinitis, allergic conjunctivitis, adult respiratory distress syndrome, chronic pulmonary inflammation, chronic obstructive pulmonary disease, chronic heart failure, silicosis, endotoxemia, toxic shock syndrome, inflammatory bowel disease, tuberculosis, atherosclerosis, depression, anxiety, sleep disorders, schizophrenia, neurodegenerative disease, Alzheimer's disease, Parkinson's disease, Huntington's disease, amyotrophic lateral sclerosis, epilepsy, multiple sclerosis, aneurism, stroke, irritable bowel syndrome, muscle degeneration, bone resorption diseases, osteoporosis, diabetes, reperfusion injury, graft vs. host reaction, allograft rejections, sepsis, systemic cachexia, cachexia secondary to infection or malignancy, cachexia secondary to acquired immune deficiency syndrome (AIDS), malaria, leprosy, infectious arthritis, leishmaniasis, Lyme disease, glomerulonephritis, gout, psoriatic arthritis, Reiter's syndrome, traumatic arthritis, rubella arthritis, Crohn's disease, ulcerative colitis, acute synovitis, gouty arthritis, spondylitis, and non articular inflammatory conditions, for example, herniated/ruptured/prolapsed intervertebral disk syndrome, bursitis, tendonitis, tenosynovitis, fibromyalgic syndrome and other inflammatory conditions associated with ligamentous sprain and regional musculoskeletal strain, pain, for example that associated with inflammation and/or trauma, osteopetrosis, restenosis, thrombosis, angiogenesis, cancer including breast cancer, colon cancer, lung cancer, prostatic cancer or multiple melanoma, which comprises administering to said subject a therapeutically effective amount of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof.

A further aspect of the invention provides a method of treatment of a human or animal subject suffering from rheumatoid arthritis, asthma, psoriasis, chronic pulmonary inflammation, chronic obstructive pulmonary disease, chronic heart failure, systemic cachexia, glomerulonephritis, Crohn's disease, neurodegenerative disease, Alzheimer's disease, Parkinson's disease, epilepsy and cancer including breast cancer, colon cancer, lung cancer and prostatic cancer, which comprises administering to said subject a therapeutically effective amount of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof.

A further aspect of the invention provides a method of treatment of a human or animal subject suffering from rheumatoid arthritis, asthma, psoriasis, chronic pulmonary inflammation, chronic obstructive pulmonary disease, chronic heart failure, systemic cachexia, glomerulonephritis, Crohn's disease and cancer including breast cancer, colon cancer, lung cancer and prostatic cancer, which comprises administering to said subject a therapeutically effective amount of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof.

A further aspect of the invention provides a method of treatment of a human or animal subject suffering from rheumatoid arthritis, asthma, chronic pulmonary inflammation, chronic obstructive pulmonary disease, neurodegenerative disease, Alzheimer's disease, Parkinson's disease and epilepsy which comprises administering to said subject a therapeutically effective amount of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof.

A further aspect of the invention provides a method of treatment of a human or animal subject suffering from any type of pain including chronic pain, rapid onset of analgesis, neuromuscular pain, headache, cancer pain, acute and chronic inflammatory pain associated with osteoarthritis and rheumatoid arthritis, post operative inflammatory pain, neuropathic pain, diabetic neuropathy, trigeminal neuralgia, post-hepatic neuralgia, inflammatory neuropathies and migraine pain which comprises administering to said subject a therapeutically effective amount of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof.

A further aspect of the invention provides a method of treatment of a human or animal subject suffering from depression (including bipolar disorders and mood disorders), anxiety (including panic attacks, phobias and obsessive compulsive disorder), sleep disorders (including hypersomnia, narcolepsy and circadian rhythm disorders) or schizophrenia (including the sub-types paranoid type, disorganised type, catatonic type, undifferentiated type and residual type) which comprises administering to said subject a therapeutically effective amount of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof.

A further aspect of the invention provides a method of treatment of a human or animal subject suffering from rheumatoid arthritis which comprises administering to said subject a therapeutically effective amount of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof.

A further aspect of the invention provides a method of treatment of a human or animal subject suffering from chronic obstructive pulmonary disease which comprises administering to said subject a therapeutically effective amount of N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof.

A further aspect of the invention provides N-(2,2-dimethylpropyl)-6-{3-fluoro-5-[(3-isoxazolylamino)carbonyl]-2-methylphenyl}-3-pyridinecarboxamide or a pharmaceutically acceptable derivative thereof for use in the treatment of a condition or disease state mediated by p38 kinase activity or mediated by cytokines produced by the activity of p38 kinase.

A further aspect of the invention provides the use of N-(2,2-dimethylpropyl)-6-{3-fluoro-5-[(3-isoxazolylamino)carbonyl]-2-methylphenyl}-3-pyridinecarboxamide, or a pharmaceutically acceptable derivative thereof, in the manufacture of a medicament for use in the treatment of a condition or disease state mediated by p38 kinase activity or mediated by cytokines produced by p38 kinase activity.

N-(2,2-Dimethylpropyl)-6-{3-fluoro-5-[(3-isoxazolylamino)carbonyl]-2-methylphenyl}-3-pyridinecarboxamide and its derivatives may be employed alone or in combination with other therapeutic agents for the treatment of the above-mentioned conditions. Combination therapies according to the present invention thus comprise the administration of N-(2,2-dimethylpropyl)-6-{3-fluoro-5-[(3-isoxazolylamino)carbonyl]-2-methylphenyl}-3-pyridinecarboxamide or a pharmaceutically acceptable salt or solvate thereof and at least one other pharmaceutically active agent.

The compound of the invention or pharmaceutically acceptable salt(s) or solvate(s) thereof and the other pharmaceutically active agent(s) may be administered together or separately and, when administered separately, this may occur separately or sequentially in any order. When combined in the same formulation it will be appreciated that the two compounds must be stable and compatible with each other and the other components of the formulation. When formulated separately they may be provided in any convenient formulation, conveniently in such manner as are known for such compounds in the art. The amounts of the compound of the invention or pharmaceutically acceptable salt(s) or solvate(s) thereof and the other pharmaceutically active agent(s) and the relative timings of administration will be selected in order to achieve the desired combined therapeutic effect. Appropriate doses will be readily appreciated by those skilled in the art. It will be appreciated that the amount of the compound of the invention required for treatment will vary with the nature of the condition being treated and the age and condition of the patient and will ultimately be at the discretion of the attendant physician or veterinarian.

In rheumatoid arthritis therapy, combination with other chemotherapeutic or antibody agents is envisaged. Suitable examples of pharmaceutically active agents which may be employed in combination with the compound of the invention and its salts and solvates for rheumatoid arthritis therapy include: immunosuppressants such as amtolmetin guacil, mizoribine and rimexolone; anti-TNFα agents such as etanercept, infliximab, diacerein; tyrosine kinase inhibitors such as leflunomide; kallikrein antagonists such as subreum; interleukin 11 agonists such as oprelvekin; interferon beta 1 agonists; hyaluronic acid agonists such as NRD-101 (Aventis); interleukin 1 receptor antagonists such as anakinra; CD8 antagonists such as amiprilose hydrochloride; beta amyloid precursor protein antagonists such as reumacon; matrix metalloprotease inhibitors such as cipemastat and other disease modifying anti-rheumatic drugs (DMARDs) such as methotrexate, sulphasalazine, cyclosporin A, hydroxychoroquine, auranofin, aurothioglucose, gold sodium thiomalate and penicillamine.

In COPD therapy, combination with other therapeutically active agents such as a 2 adrenoreceptor agonist, an anti-histamine, an anti-allergic agent, an anti-inflammatory agent (including a steroid), an anticholinergic agent or an antiinfective agent is envisaged. Suitable examples of pharmaceutically active agents which may be employed in combination with the compound of the invention and its salts and solvates for COPD therapy include: β₂-adrenoreceptor agonists such as salmeterol (e.g. as racemate or a single enantiomer such as the R-enantiomer), salbutamol, formoterol, salmefamol, fenoterol or terbutaline and salts thereof, for example the xinofoate salt of salmeterol, the sulphate salt or free base of salbutamol or the fumarate salt of formoterol; anti-inflammatory steroids such as fluticasone propionate and budesonide; anticholinergic agents such as ipratropium bromide, oxitropium bromide or tiotropium bromide; non-steroidal anti-inflammatory (NSAID) drugs such as a leukotriene antagonist (e.g. montelukast), an iNOS inhibitor, a tryptase inhibitor, an elastase inhibitor, a beta-2 integrin antagonist, an adenosine a2a agonist, a chemokine antagonist such as a CCR3 antagonist and a 5-lipoxygenase inhibitor; or an antiinfective agent such as an antibiotic or an antiviral.

### EXAMPLE

The following example is an illustrative embodiment of the invention, not limiting the scope of the invention in any way. Reagents are commercially available or are prepared according to procedures in the literature.

LCMS was conducted on a column (3.3 cm×4.6 mm ID, 3 um ABZ+PLUS), at a Flow Rate of 3 ml/min, Injection Volume of 5 μl, at room temperature and UV Detection Range at 215 to 330 nm. Solvent A: 10 mM Aqueous ammonium acetate+0.1% formic acid. Solvent B: 95% Acetonitrile+0.05% formic acid. Gradient: 0% A/0.7 min, 0-100% A/3.5 min, 100% A/0.1 min, 100-0% A/0.2 min.

### Intermediate 1: 3-Fluoro-5-iodo-4-methylbenzoic Acid

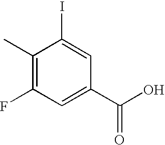

3-Fluoro-4-methylbenzoic acid (182 g) was added to trifluoromethanesulphonic acid (1.12 L) and the solution cooled to −20° C. under nitrogen. Iodosuccinimide (266 g) was added in portions over 75 min, maintaining a reaction temperature of −18 to −19° C., and the reaction was then stirred at −20° C. for 4 h. Iodosuccinimide (54.8 g) was added portionwise and the reaction stirred at −20° C. overnight. Iodosuccinimide (19 g) added before stirring at −20° C. for a further 24 h. The reaction was warmed to −5° C. and the suspension poured into a stirred mixture of ice (3 kg) and sodium thiosulphate solution (10%). The mixture was filtered and the solid partially dried on the sinter. The solid was partitioned between ethyl acetate (5 L) and aqueous sodium thiosulphate solution (10%, 1.5 L) and the organic phase was washed with sodium thiosulphate solution (10%) dried (sodium sulphate) and concentrated under vacuum to ca. 600 ml. The resulting slurry was allowed to stand for 4 h and the solid was collected by filtration, washed with ethyl acetate and dried, to give the title compound (215 g).

LC-MS: Rt 3.75 min.

### Intermediate 2: Methyl 3-fluoro-5-iodo-4-methylbenzoate

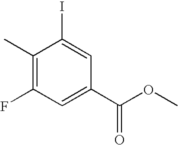

A mixture of 3-fluoro-5-iodo-4-methylbenzoic acid (Intermediate 1, 28 g) and thionyl chloride (40 ml) was heated at reflux for 3 h, before the reaction was allowed to cool and the excess thionyl chloride was removed under vacuum, azeotroping with DCM (3×50 ml). The remaining oil was dissolved in DCM (50 ml) and added dropwise with stirring to a solution of dry methanol (30 ml) and triethylamine (20 ml) in DCM (150 ml) with ice/water cooling. The reaction was stirred at room temperature overnight, diluted with DCM (100 ml) then washed with water (200 ml), hydrochloric acid (2M, 2×200 ml), water (200 ml) and brine (100 ml). The organic phase was dried (magnesium sulphate) and reduced to dryness under vacuum. The residual oil was purified by chromatography on a silica gel column eluting with DCM/hexane (10-20% DCM) to give the title compound (30.3 g).

NMR: CDCl₃ δH 8.27, (1H, s) 7.65, (1H, dd) 3.92, (3H, s) 2.41, (3H, d).

### Intermediate 3: Methyl 3-fluoro-4-methyl-5-(4,4,5,5-tetramethyl-1,3,2-dioxaborolan-2-yl)benzoate

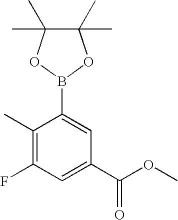

Potassium acetate (50.5 g) was added to a solution of methyl 3-fluoro-5-iodo-4-methylbenzoate (Intermediate 2, 30.3 g) in DMF (300 ml). Bis(pinnacolato)diboron (39.4 g) was added to the mixture and the mixture was degassed. Pd(dppf)Cl₂ (0.85 g) was added to the mixture and the reaction was heated at 80-85° C. for 18 h, allowed to cool and the DMF was removed under vacuum. The residue was partitioned between ethyl acetate (400 ml) and water (250 ml) and the aqueous layer was further extracted with ethyl acetate (300 ml). The combined organic phases were washed with hydrochloric acid (2M, 3×150 ml) aqueous lithium chloride solution (10%, 2×200 ml) and brine (200 ml) before drying over magnesium sulphate. The ethyl acetate was evaporated under vacuum and the residue was triturated with DCM (100 ml). The white precipitate was collected by filtration and washed with DCM. The solid was recrystallised from acetonitrile to give the title compound (5.42 g). The DCM fraction was reduced to dryness under vacuum and triturated with DCM (70 ml), to give further material (3.32 g). The DCM fraction was further purified by flash chromatography on silica, eluting with 10% ethyl acetate in cyclohexane. The combined product fractions were recrystallised from acetonitrile (200 ml) to give further title compound (14.7 g). (combined yield 23.44 g).

LC-MS: Rt 3.84 min.

### Intermediate 4: 6-Chloro-N-(2,2-dimethylpropyl)-3-pyridinecarboxamide

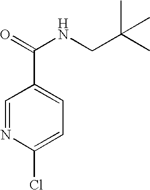

A solution of neopentylamine (125 ml) in dichloromethane (400 ml) was treated with sodium carbonate (175 g) and the stirred mixture was cooled to 5° C. A suspension of chloronicotinyl chloride (175 g, from Avocado) in dichloromethane (400 ml) was added portionwise and the mixture was stirred for 1 h, warmed to room temperature and then stirred for a further 2 h. The mixture was partitioned between water and dichloromethane, the phase were separated and the aqueous layer was re-extracted with dichloromethane. The combined organic extracts were washed with brine, dried over sodium sulphate then concentrated under vacuum to give the title compound (216.9 g).

LC-MS: Rt 2.67 min.

### Intermediate 5: 3-(5-{[(2,2-Dimethylpropyl)amino]carbonyl}-2-pyridinyl)-5-fluoro-4-methylbenzoic Acid

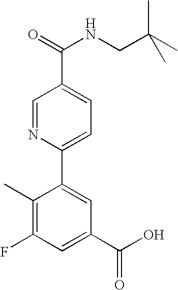

A suspension of methyl 3-fluoro-4-methyl-5-(4,4,5,5-tetramethyl-1,3,2-dioxaborolan-2-yl)benzoate (Intermediate 3, 3.89 g), 6-chloro-N-(2,2-dimethylpropyl)-3-pyridinecarboxamide (Intermediate 4, 3.00 g) and tetrakis(triphenylphosphine)palladium (305 mg) in a mixture of aqueous sodium hydrogen carbonate (1M, 41 ml) and isopropanol (55 ml) was degassed using a stream of argon for 20 min. The flask was then sealed, heated overnight at 85° C. then stirred at room temperature for 24 h. Aqueous sodium hydrogen carbonate (1M, 15 ml) was added and the mixture was concentrated under vacuum. The residue was partitioned between ethyl acetate and aqueous sodium hydrogen carbonate (1M) the layers were separated and the aqueous phase was neutralised with hydrochloric acid (2M). The resultant precipitate was collected by filtration, washed with water and dried to give a small amount of title compound (0.74 g). The organic layer was washed with water and brine, dried then concentrated to give the methyl ester (4.38 g). The ester was suspended in methanol (30 ml) aqueous sodium hydroxide solution (2M, 20 ml) was added and the mixture was stirred overnight at room temperature. The solvent was removed under vacuum and the residue was partitioned between water and dichloromethane. The aqueous layer was separated and acidified to pH5 using hydrochloric acid (2M) On cooling the resulting precipitate was collected by filtration, washed with water and dried to give the title compound (2.77 g).

TLC: (silica) dichloromethane:methanol 9:1 R_{f}=0.15.

### Intermediate 6: 3-Fluoro-5-iodo-N-3-isoxazolyl-4-methylbenzamide

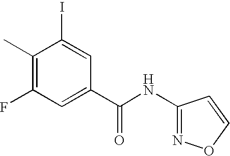

Thionyl chloride (10.4 ml) was added to 3-fluoro-5-iodo-4-methylbenzoic acid (Intermediate 1, 10 g) and the mixture was stirred at 95° C. for 1.75 h. The thionyl chloride was removed under vacuum, the residue was azeotroped with toluene and the residue was dissolved in dry dichloromethane (100 ml). A solution of 3-isoxazolamine (3.6 g) N,N-diisopropylethylamine (12.4 ml) and 4-dimethylaminopyridine (4.36 g) in dry dichloromethane (250 ml) was stirred at room temperature for 45 min then the acid chloride solution was added over a period of about 20 min. The reaction mixture was stirred at room temperature for 3 h, water was added and the mixture was partitioned between ethyl acetate and hydrochloric acid (1M). The organic layer was separated, the aqueous layer was re-extracted with ethyl acetate and the combined organic extracts were washed with aqueous sodium hydrogen carbonate (1M) and brine, dried and concentrated under vacuum to give the title compound as a cream solid (11.9 g).

LC-MS: Rt 1.80 min.

### Intermediate 7: {3-Fluoro-5-[(3-isoxazolylamino)carbonyl]-2-methylphenyl}boronic acid

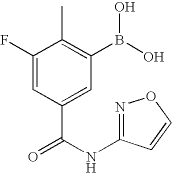

A solution of 3-fluoro-5-iodo-N-3-isoxazolyl-4-methylbenzamide (Intermediate 6, 13.8 g) in dry tetrahydrofuran (250 ml) at −12° C. under nitrogen was treated, over 9 min, with a solution of isopropylmagnesium chloride in tetrahydrofuran (2M, 49.8 ml). The reaction mixture was allowed to warm to 0° C., stirred for 1.75 h, cooled to −10° C. then treated with triisopropylborate (13.8 ml) over 5 min. The cooling bath was removed and the reaction mixture was stirred at room temperature overnight. Water (50 ml) was added, the mixture was concentrated under vacuum to ca. 150 ml and the residue was acidified (to pH1) using concentrated hydrochloric acid, yielding a white precipitate. The mixture was extracted with ethyl acetate (×2) and the organic layer was dried and concentrated under vacuum. The resulting solid was triturated with ether (ca150 ml) then collected by filtration to give the title compound as a pale peach solid (5.31 g).

LC-MS: Rt 1.28 min.

### Example 1

### N-(2,2-Dimethylpropyl)-6-{(3-fluoro-5-[(3-isoxazolylamino)carbonyl]-2-methylphenyl}-3-pyridinecarboxamide

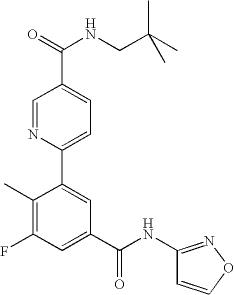

### Method 1

Oxalyl chloride (258 mg, 2.03 mmol) was added dropwise to a suspension of 3-(5-{[(2,2-dimethylpropyl)amino]carbonyl}-2-pyridinyl)-5-fluoro-4-methylbenzoic acid (Intermediate 5, 350 mg) in dichloromethane (3 ml) then stirred in an ice bath at 0° C. N,N-Dimethylformamide (4 or 5 drops) was added and the mixture was stirred at 0° C. for 10 min; the cooling bath was then removed and stirring was continued for a further 15 min. The reaction mixture was concentrated under vacuum at room temperature and the residue was dissolved in dichloromethane (4 ml). 3-Isoxazolamine (94 mg) and triethylamine (0.28 ml) were added and the reaction mixture was stirred overnight at room temperature. Water (5 ml) was added and the layers were separated. The aqueous layer was re-extracted with dichloromethane and the combined organic extracts were washed with brine (5 ml), dried (MgSO₄), filtered and concentrated. The crude product (400 mg) was purified by dry flash chromatography (silica, ethyl acetate:hexanes 1:1) to giving the title compound (165 mg).

TLC: (silica) hexanes:ethyl acetate 7:8 R_{f}=0.14.

### Method 2a

A mixture of {3-fluoro-5-[(3-isoxazolylamino)carbonyl]-2-methylphenyl}boronic acid (Intermediate 7, 0.5 g) 6-chloro-N-(2,2-dimethylpropyl)-3-pyridinecarboxamide (Intermediate 4, 0.43 g) tetrakis(triphenylphospine)palladium (44 mg) and aqueous sodium hydrogen carbonate (1M, 3.78 ml) in isopropanol (7 ml) in a sealed vial was heated at 110° C. in a microwave oven for 20 min. The cooled mixture was partitioned between water and ethyl acetate, the phases were separated and the aqueous layer was re-extracted with ethyl acetate. The combined organic extracts were washed with brine, dried and concentrated under vacuum. The residue was purified by column chromatography on silica eluting with dichloromethane/ethyl acetate (100:0 to 50:50) to give the title compound as an off-white solid (0.44 g).

LC-MS: Rt 3.14 min, MH+411.

### Method 2b

6-Chloro-N-(2,2-dimethylpropyl)-3-pyridinecarboxamide (Intermediate 4, 9.0 g) was added to {3-fluoro-5-[(3-isoxazolylamino)carbonyl]-2-methylphenyl}boronic acid (Intermediate 7, 7.72 g) in nitrogen-purged isopropanol (130 ml). Aqueous sodium hydrogen carbonate (1M, 68 ml) and tetrakis(triphenylphosphine)palladium (0.78 g) were added and the reaction mixture was stirred at 80° C. for 3 h. The cooled mixture was partitioned between water and ethyl acetate, the phases were separated and the aqueous layer was re-extracted with ethyl acetate. The combined organic extracts were washed with water and brine, dried and concentrated under vacuum. The residue was purified by column chromatography on silica eluting with dichloromethane/ethyl acetate (3:1) to give a pale yellow solid. Recrystallisation from ethyl acetate gave the title compound as a white solid (4.9 g).

LC-MS: Rt 3.13 min, MH+411.

### Method 3

The compound of the present invention was also prepared in the following way in accordance with Scheme 3 described above.

### Preparation of 3-Fluoro-5-iodo-4-methylbenzoic acid

3-Fluoro-4-methylbenzoic acid (1 eqv) and potassium chloride (0.01 eqv) were added to trifluoromethanesulphonic acid (7 vol), then the resulting solution was cooled to −20° C. N-iodosuccinimide (1.3 eqv) was added portionwise over 3 h, then the mixture stirred at −20° C. for 11 h. The mixture was then warmed to 20° C. over 1.5 h, then held at this temperature for 1 h. The resulting reaction mixture was added over 1.75 h to a mixture of water (22 vol) and sodium sulfite 10% aq. soln. (7.5 vol) at 5 to +15° C.

The resulting suspension was filtered and the cake washed with water (3×3 vol). After drying the solid product under a stream of nitrogen, the solid was dissolved in ethyl acetate (13.6 vol), then the resulting solution was washed with 10% aqueous sodium sulphate (2×4 vol), with the aqueous washed then back-extracted with ethyl acetate (5 vol). The combined ethyl acetate solutions were washed with brine (4.9 vol), then concentrated to about 3.5 vol. The resulting suspension was cooled to 0-5° C. over 1 h. The solid was collected on a filter, then dried under vacuum at 45-50° C. to give the title compound.

A second crop of product was obtained by added methylcyclohexane (2 vol) to the crystallisation liquors, concentrating to a volume of 3.3 vol, then adding further methylcyclohexane (3.3 vol). The resulting suspension was cooled to −3° C. over 1 h, and the solid was collected on a filter, then dried on the filter. Total recovery: 62% th.

### Preparation of methyl 3-fluoro-5-iodo-4-methylbenzoate

3-Fluoro-5-iodo-4-methylbenzoic acid (1 eqv) was dissolved in methanol (6.6 vol) and treated with 98% sulphuric acid (0.375 vol) at 20° C. The solution was heated to 50° C. and stirred for 15 h at this temperature then the solution cooled to 20° C.

The volume of the solution was reduced to 25% of the initial volume by vacuum distillation. After cooling to 20° C., TBME (5.5 vol) was added, followed by 8% aqueous NaHCO₃ solution (9.4 vol) over 15 min to a pH of 7-8. After stirring for 10 min, the layers were separated and the aqueous layer was extracted with further TBME (5.5 vol). After stirring for 10 min, the layers were separated and the combined organic layers were stirred and washed with water (4.6 vol). After stirring for 10 min, the layers were separated and the organic layer was concentrated and dried at 45-50° C. to give the title compound. Yield: ca. 100% th.

### Preparation of {3-fluoro-2-methyl-5-[(methyloxy)carbonyl]phenyl}boronic acid

Methyl 3-fluoro-5-iodo-4-methylbenzoate (1 eqv) was dissolved in THF (2.9 vol) at 20° C. and then cooled to −25° C. Isopropylmagnesium chloride 2 M in THF (1.3 eq., 2.22 vol) was added at −25° C. over 25 min then the mixture was stirred at −25° C. for a further 1 h. The resulting solution was added over 10 min at −25° C. to a solution of triisopropyl borate (2 eq., 1.56 vol) in THF (6.59 vol). The orange colored solution was stirred for 1 h at −25 to −21° C., then it was warmed to +20° C. over 2 h.

The mixture was cooled to 0° C. then added to 7.1% aqueous HCl 7.1% (4.2 vol) at 0-5° C. The mixture was warmed to 20° C. over about 30 min and stirred for a further 30 min at 20° C. the resulting pH should be <3. The layers were separated and TBME (6 vol) was added to the aqueous layer. The mixture was stirred for 15 min and the layers were separated. The combined organic layers were washed with brine (3.75 vol) then the layers were separated. The organic layer was washed with water (2.9 vol) then the mixture was stirred for 15 min and the layers were separated.

The organic layer was concentrated at 55° C. under vacuum to 25% of its volume. Then heptane (6.6 vol) was added. The mixture was concentrated at 55° C. under vacuum to 30% of its volume, then the mixture was cooled to 25° C., then further to 0° C. over 30 min. After stirring for 2 h at this temperature, the precipitated material was filtered and the filter cake was washed with cold heptane (2 vol). The filter cake was dried on the filter for 2 h at 20° C. to give the title compound. Yield: about 76% th.

### Preparation of methyl 3-(5-{[(2,2-dimethylpropyl)amino]carbonyl}-2-pyridinyl)-5-fluoro-4-methylbenzoate

To a mixture of {3-Fluoro-2-methyl-5-[(methyloxy)carbonyl]phenyl}boronic acid. (1 eqv), and 6-chloro-N-(2,2-dimethylpropyl)-3-pyridinecarboxamide (1.05 eqv) in IPA (9 vol) at 20° C., 7.4% aqueous NaHCO₃ solution (4.4 vol) was added to form a suspension, tetrakis(triphenylphosphine)palladium (0.01 eq, 0.045 wt) was added. The suspension was heated to 80° C. over 1 h and stirred for 17 h at this temperature.

The mixture was cooled to 20° C. and water (1.7 vol) was added. The mixture was concentrated under vacuum to 50% of its volume. After cooling to 20° C., ethyl acetate (6.4 vol) was added. The mixture was stirred for 10 min, then the layers were separated. The aqueous layer was extracted again with ethyl acetate (6.4 vol). The combined organic layers were washed with water (5 vol), and brine (2.5 vol), then the organic layer was concentrated under vacuum to give the title compound. Yield: 95% th.

### Preparation of 3-(5-{[(2,2-dimethylpropyl)amino]carbonyl}-2-pyridinyl)-5-fluoro-4-methylbenzoic Acid

Methyl 3-(5-{[(2,2-dimethylpropyl)amino]carbonyl}-2-pyridinyl)-5-fluoro-4-methylbenzoate (1 eqv), THF (4 vol) and water (2.5 wt) were stirred at 20° C. and then a solution of lithium hydroxide (2 eqv) in water (2 vol) was added at 5-7° C. over 15 min. After warming to 20° C. over 30 min, the mixture was stirred for 13 h at 20° C. Approximately 85% of the THF in the mixture was removed by vacuum distillation, then dichloromethane (10 vol) and water (1.7 vol) were added. The mixture was stirred for 5 min, then the layers were separated. The aqueous layer was washed again with dichloromethane (1.7 vol) then the layers were separated. At 5° C., the aqueous layer was acidified using 5M HCl (2 eq.), forming a suspension which was stirred for a further 30 min at 5° C. The precipitated product was collected by filtration and the cake was washed with cold water (2 vol), then dried on the filter by passing over a stream of nitrogen.

The wet cake was heated to reflux in acetone (4 vol) for 1 h then was cooled to 0° C. over 3 h. The solid product was collected by filtration, then washed with cold acetone (0° C., 2×0.8 vol). The solid was dried on the filter under a stream of nitrogen to give the title compound. Yield: 76% th

### Preparation of 6-Chloro-N-(2,2-dimethylpropyl)-3-pyridinecarboxamide

6-Chloro-3-pyridinecarbonyl chloride (1 eqv) was placed in the reactor and dissolved in dichloromethane (2.27 vol). The solution was transferred in a feeding tank. A mixture of sodium carbonate (1.66 eq,) dichloromethane (5.11 vol) and neopentylamine (1.07 eqv) was stirred and cooled to 0° C. A solution of 6-chloro-3-pyridinecarbonyl chloride (1 eqv) in dichloromethane (2.27 vol) was added over 90 min. After stirring for further 15 min at 0-5° C., the suspension was warmed to 20° C. over 16 h.

Water (9.7 vol) was added then the organic layer was separated and the aqueous layer was extracted with dichloromethane (2.84 vol). The combined organic layers were washed with brine (2.84 vol). The mixture was concentrated under vacuum, then at approximately 35° C., heptane (16.5 vol) was added. The resulting suspension was cooled to 0-5° C. over 1 h and stirred for 2 h at 0° C. The solid was filtered and washed with cold heptane (2.84 vol), then dried at 20° to give the title compound. Yield: 95%.

### Preparation of N-(2,2-dimethylpropyl)-6-{3-fluoro-5-[(3-isoxazolylamino)carbonyl]-2-methylphenyl}-3-pyridinecarboxamide

A solution of 3-(5-{[(2,2-dimethylpropyl)amino]carbonyl}-2-pyridinyl)-5-fluoro-4-methylbenzoic acid (1 eqv) in dichloromethane (22 vol) was distilled to remove 50% of the solvent. DMF (0.034 vol) was added and the mixture was cooled to 0° C. Thionyl chloride (2.5 eqv) was added at 0-8° C. over 10 min, then the suspension was heated to 50° C. and stirred for 15 h at this temperature. Thionyl chloride was partially removed by vacuum distillation and then toluene (6 vol) was added and 8.5 vol of a thionyl chloride/toluene mixture was distilled under vacuum, then the mixture was cooled to 20° C. and dichloromethane (22 vol) was added.

A mixture of 3-isoxazolamine (1.2 eqv), dichloromethane (1 vol), DIPEA (1 eq.) and DMAP (1 eq.) was cooled to 0° C., then the acid chloride solution in dichloromethane was slowly added at 0-5° C. over 1 h. The mixture was warmed to 20° C., then stirred for 16 h at this temperature.

The mixture was stirred for 5 min with 1M sulphuric acid (4 vol), to pH 1, then the layers were separated and the organic layer was washed with water (9 vol), then methanol (3.5 vol) was added to the organic layer, which was then washed with saturated aqueous NaHCO₃ (8 vol) to pH 9. The organic layer was then washed with water (10 vol).

The combined aqueous layers were extracted with a solution of dichloromethane (10 vol) and methanol (0.85 vol), then combined organic layers were concentrated to 30% of the initial volume under vacuum. The concentrate was cooled to 0° C., then stirred at this temperature for 2 h. The precipitated product was filtered and washed with cold dichloromethane (4.5 vol). The filter cake was dried on the filter under nitrogen.

The crude product was heated to reflux with acetone (9 vol) and water (9 vol) was added to the solution over 5 min. After stirring for a further 25 min at 50-60° C., the mixture was slowly cooled to 5° C. over 3 h. The precipitated product was filtered, washed with cold water (2.5 vol) then dried at 50° C. under vacuum to give the title compound. Yield: 61% th.

### Example 2

### Preparation of Polymorphic forms of N-(2,2-dimethylpropyl)-6-{3-fluoro-5-[(3-isoxazolylamino)carbonyl]-2-methylphenyl}-3-pyridinecarboxamide

### Form 1

The crystalline product from Methods 1-3 described above was characterised by one or more of the methods described in Example 3 and was designated as Form 1.

### Form 2

A solvate of the compound of the invention was prepared by suspending 300 mg of Form 1 of the compound in a total of 6.5 ml chloroform (4.5 ml was initially added followed by a further 2 ml) and the resulting suspension was then subjected to temperature cycling from 0° C. to 40° C. in 1 hour blocks for approximately 72 hours (hereafter referred to as the “the chloroform solvate”).

A hot stage microscopy experiment was then performed on the chloroform solvate using a Linkum LT350 hot stage viewed through a Olympus BX41 microscope. The sample was heated at 10 degrees per minute until 200° C. at which time the heating was stopped and the sample allowed to cool to ambient over approximately 10 minutes. Polarised Light Microscopy was then performed on the melt-cooled material. Material had clearly crystallised out of the melt and this crystallised material was used to seed a slurry of approximately 75 mg of Form 1 material in 1 ml of ethyl acetate which was held at 60° C. in overnight. The solid was filtered off, and characterised by methods described in Example 3. These data were sufficient to designate this polymorph as anhydrous Form 2.

### Form 3

A Differential Scanning Calorimetry temperature cycling experiment was performed on a sample of the chloroform solvate. The sample was prepared in an aluminium pan with non pin holed lid, heated to 204° C. at 10° C. per minute, cooled to ambient over approximately 200 seconds and then heated up to 250° C. at 10° C. per minute. The resulting material was characterised by methods described in Example 3. These data were sufficient to designate this polymorph as anhydrous Form 3.

### Form 4

A thin slurry of approximately 100 mg of Form 1 was formed at 60° C. in approximately 1.5 ml of ethyl acetate. The resulting slurry was then heated to 70° C. over a time period of approximately 30 seconds. A clear solution was formed and the solution held at this temperature for approximately 10 minutes. The solution was then cooled to 50° C., and seeded with a few milligrams of Form 3 prepared as described above. This solution was then cooled to 40° C., and 10 minutes later cooled to 30° C. The resulting solid was isolated by filtration and characterised by methods described in Example 3. These data were sufficient to designate this polymorph as anhydrous Form 4.

### Form 5

A Differential Scanning Calorimetry temperature cycling experiment was performed on a sample of Form 4 prepared as described above. The sample was prepared in an aluminium pan with non pin holed lid, heated to 217° C. at 10° C. per minute, cooled to ambient over approximately 200 seconds and then heated up to 250° C. at 10° C. per minute. The resulting material was characterised by methods described in Example 3. These data were sufficient to designate this polymorph as anhydrous Form 5.

### Example 3

### Characterisation of the Polymorphic Forms of N-(2,2-dimethylpropyl)-6-{3-fluoro-5-[(3-isoxazolylamino)carbonyl]-2-methylphenyl}-3-pyridinecarboxamide

### X-Ray Powder Diffraction (XRPD)

FIGS. 1-5 show the diffraction pattern for each of the polymorphic forms 1-5 respectively.which were obtained using copper Kα radiation on a PANalytical X'Pert Pro diffractometer, model PW3040/60, serial number DY1850 equipped with a PANalytical X'Celerator detector. The sample was dispersed onto a zero background holder and scanned from 2 to 40 ° 2θ using the following acquisition parameters: 45 mA, 40 kV, 0.017 ° 2θ step, 32 s step time. The sample was spun at 25 rpm during analysis.

It will be appreciated that identification of a particular polymorphic form of the compound of this invention is based primarily on observed 2 theta angle rather than on relative peak intensities. Table 1 shows the main degrees 2 theta peaks observed for each of Forms 1-5 with the characteristic peaks shown with a grey fill. The margin of error is approximately ±0.1 for each of the peak assignments.

TABLE 1

| Main | | | | Main |
|---|---|---|---|---|
| peaks for | Main peaks for | Main peaks for | Main peaks for | peaks for |
| Form 1 | Form 2 | Form 3 | Form 4 | Form 5 |
| 3.0 | 10.2 | 5.8 | 5.7 | 5.7 |
| 6.0 | 14.5 | 9.2 | 11.5 | 11.5 |
| 8.9 | 15.8 | 9.4 | 12.2 | 16.1 |
| 10.2 | 15.9 | 9.6 | 12.3 | 18.0 |
| 11.3 | 16.8 | 10.8 | 14.5 | 18.6 |
| 11.9 | 18.0 | 11.6 | 19.0 | 21.0 |
| 13.0 | 19.6 | 15.9 | 28.2 | 21.4 |
| 13.5 | 20.7 | 19.5 | — | 26.5 |
| 15.0 | 22.1 | 21.7 | — | — |
| 15.1 | 22.8 | 21.9 | — | — |
| 17.5 | 25.6 | — | — | — |
| 20.0 | 30.1 | — | — | — |
| 25.0 | — | — | — | — |
| 27.0 | — | — | — | — |

### Differential Scanning Calorimetry (DSC)

DSC was performed on either a TA Instruments Q1000 (instrument number: 970001.901, serial number: 1000-0126) or M2920 (instrument number: 915001.901, serial number: M2920-234) Differential Scanning Calorimeter equipped with a refrigerated cooling system. The sample was heated in a loosely covered aluminium pan from 25-350° C. using a heating rate of 10° C./min. Forms 1 and 2 each show distinct endothermic melts followed by thermal decomposition.

The DSC thermograms of Forms 1 and 2 are detailed in Table 2.

TABLE 2

| | | | Temperature, | Heat of |
|---|---|---|---|---|
| Form | Event | Interpretation | ° C. | Fusion, J/g |
| 1 | Endotherm | Melt | 187 | 93 |
| 2 | Endotherm | Melt | 203 | 103 |

The margin of error is approximately ±2° C. for the peak maximum and ±5 J/g for the heat of fusion.

### ABBREVIATIONS

Aq Aqueous
DCM Dichloromethane
DIPEA N,N-Diisopropylethylamine
DMAP 4-Dimethylaminopyridine
DMF Dimethylformamide
Et Ethyl
H Hours
iPr Isopropyl
Me Methyl
Min Minutes
NIS N-Iodosuccinimide
Pd(dppf)Cl_{2 [}1,1′-bis(Diphenylphosphino)ferrocene]dichloropalladium (II) complex with dichloromethane (1:1)
Pd(PPh₃)₄ Tetrakis(triphenylphosphine)palladium
TBME Tert-butyl methyl ether
THF Tetrahydrofuran

### BIOLOGICAL EXAMPLES

The activity of the compound of the invention as a p38 inhibitor may be determined by the following assays:

### Assay 1: Fluorescence Anisotropy Kinase Binding Assay

The kinase enzyme, fluorescent ligand and a variable concentration of test compound are incubated together to reach thermodynamic equilibrium under conditions such that in the absence of test compound the fluorescent ligand is significantly (>50%) enzyme bound and in the presence of a sufficient concentration (>10×Kⱼ) of a potent inhibitor the anisotropy of the unbound fluorescent ligand is measurably different from the bound value.

The concentration of kinase enzyme should preferably be 2×K_{f}. The concentration of fluorescent ligand required will depend on the instrumentation used, and the fluorescent and physicochemical properties. The concentration used must be lower than the concentration of kinase enzyme, and preferably less than half the kinase enzyme concentration.

Recombinant human p38α was expressed as a GST-tagged protein. To activate this protein, 3.5 μM unactivated p38α was incubated in 50 mM Tris-HCl pH 7.5, 0.1 mM EGTA, 0.1% 2-mercaptoethanol, 0.1 mM sodium vanadate, 10 mM MgAc, 0.1 mM ATP with 200 nM MBP-MKK6 DD at 30 degrees for 30 mins. Following activation p38α was re-purified and the activity assessed using a standard filter-binding assay.

A typical protocol is:

All components are dissolved in buffer of composition 62.5 mM HEPES, pH 7.5, 1.25 mM CHAPS, 1 mM DTT, 12.5 mM MgCl₂ with final concentrations of 12 nM p38α and 5 nM fluorescent ligand. 30 μl of this reaction mixture is added to wells containing 1 μl of various concentrations of test compound (0.28 nM-16.6 μM final) or DMSO vehicle (3% final) in NUNC 384 well black microtitre plate and equilibrated for 30-60 mins at room temperature. Fluorescence anisotropy is read in Molecular Devices Acquest (excitation 485 nm/emission 535 nm).

Definitions: Kᵢ=dissociation constant for inhibitor binding

K_{f}=dissociation constant for fluorescent ligand binding

The fluorescent ligand is the following compound:

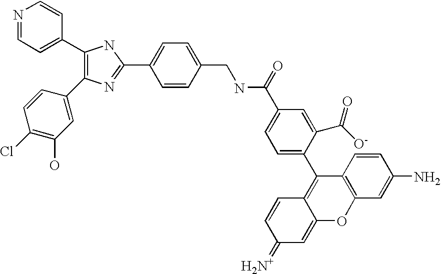

which is derived from 5-[2-(4-aminomethylphenyl)-5-pyridin-4-yl-1H-imidazol-4-yl]-2-chlorophenol and rhodamine green.

### Assay 2: TNFα Production in Human Whole Blood

p38 Mitogen-activated protein (MAP) kinase (p38) regulates the biosynthesis of pro-inflammatory cytokines such as TNFα. The potency as an inhibitor of cytokine production can thus be assessed by measuring the effects on TNFα production in LPS-stimulated leukocytes. Carrying the assay out on whole blood rather than isolated leukocytes gives a measure of whole cell potency of the compound in the presence of plasma protein and cells, particularly red blood cells that in vivo are likely to lower circulating free drug concentrations. In the assay compound is preincubated with human whole blood for an hour before cytokine production from leukocytes present in the blood is stimulated with bacterial lipopolysaccharide (LPS). After 20 h plasma is removed and assayed for the presence of TNF. A shorter incubation for the TNF generation phase of the assay (4 h) is possible but inhibition curves are more well defined and reproducible with the 20 h assay, similar observations have been reported for PDE4 inhibitors (Brideau et al 1999, B. J. Pharmacology, 126, 979-988). The TNF assay is a sandwich immunoassay using electrochemiluminescence detection technology. The TNF is captured by biotinylated anti TNF antibody immobilised on streptavidin coated magnetic beads. A ruthenium tagged secondary antibody is also bound to the TNF and the beads are then drawn into a flow cell where the beads are captured onto the surface of an electrode. The ruthenium in close proximity to the electrode is excited to emit light an the level of light emitted is proportional to the amount of TNF immobilised on the bead. The concentrations of TNF in the original whole blood assay supernatants can then be determined from a standard curve generated using authentic human TNFα.

Heparinised blood drawn from normal volunteers was dispensed (100 μl) into microtitre plate wells containing 0.5 or 1.0 μl of an appropriately diluted compound solution. After 1 hr incubation at 37° C., 5% CO2 25 μl LPS solution (S. typhosa) in RPMI 1640 (containing 1% L-glutamine and 1% Penicillin/streptomycin) was added (50 ng/ml final). The samples were incubated at 37° C., 5% CO₂ for 20 hours, 100 μls physiological saline (0.138% NaCl) was added and diluted plasma was collected using a Platemate or Biomek FX liquid handling robot after centrifugation at 1300 g for 10 min. Plasma TNFα content was determined by electrochemiluminescence assay using the IGEN technology.

Analysis may be carried out using either of the methods described below.

### Analytical Assay Method (a)

50 μl supernatant from whole blood assay plates was transferred to a 96 well polypropylene plate, each plate also contained a TNFα standard curve (0-30000 pg/ml: R+D Systems, 210-TA). 50 μl of streptavidin bead/biotinylated anti-TNFα antibody mix, 25 μl ruthenium tagged anti-TNFα monoclonal and 100 μl PBS containing 0.1% bovine serum albumin were added to each well and the plates were sealed and shaken for 2 hours before being read on an IGEN instrument.

### Analytical Assay Method (b)

TNF assay plates (MSD: cat L41IB-1) were blocked overnight with 20 μl human serum cytokine diluent (MSD). Supernatant from whole blood (40 μl) or PBMC assays (20 μl) was then added, each plate also contained a TNFa standard curve (0-10000 or 30000 pg/ml: R+D Systems, 210-TA). 20 μl TNFa detection antibody (1 μg/ml: MSD) was added and plates incubated with shaking for 2 hours at room temperature. The whole blood plates were then washed 4× with PBS/Tween 20 (0.05% v/v) and blotted dry. 150 μl 2× read buffer T (MSD Cat R92TC-1) was added to the whole blood plates and 90 μl 2.5× read buffer P (MSD Cat R92PC-1) to the PBMC plates and all plates read in the MSD Sector Imager 6000 electrochemiluminescence reader.

### Results

The compound described in the Example was tested as described above and had an IC₅₀ value of <50 nM.

The application of which this description and claims forms part may be used as a basis for priority in respect of any subsequent application. The claims of such subsequent application may be directed to any feature or combination of features described herein. They may take the form of product, composition, process or use claims and may include, by way of example and without limitation, one or more of the following claims:

1. N-(2,2-Dimethylpropyl)-6-{3-fluoro-5-[(3-isoxazolylamino)carbonyl]-2-methylphenyl}-3-pyridinecarboxamide, or a pharmaceutically acceptable derivative thereof.

2. The compound according to claim 1 which is N-(2,2-dimethylpropyl)-6-{3-fluoro-5-[(3-isoxazolylamino)carbonyl]-2-methylphenyl}-3-pyridinecarboxamide.

3. A pharmaceutical composition comprising a compound according to claim 1, or a pharmaceutically acceptable derivative thereof, in association with one or more pharmaceutically acceptable excipients, diluents and/or carriers.

4. A method for the treatment of a condition or disease state mediated by p38 kinase activity or mediated by cytokines produced by the activity of p38 kinase comprising administering to a subject in need thereof an effective amount of a compound according to claim 1, or a pharmaceutically acceptable derivative thereof.

5. (canceled)

6. A compound according to claim 1, or a pharmaceutically acceptable derivative thereof, for use in the treatment of a condition or disease state mediated by p38 kinase activity or mediated by cytokines produced by the activity of p38 kinase.

7. A method of treatment of a condition or disease state mediated by p38 kinase activity or mediated by cytokines produced by the activity of p38 kinase comprising administering to a subject in need thereof an effective amount of a compound according to claim 2.

8. A process for preparing a compound according to claim 1, or a pharmaceutically acceptable derivative thereof, which comprises:
(a) reacting a compound of formula (I) 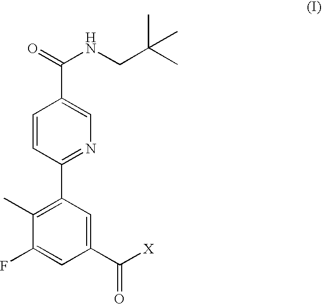
in which X is a leaving group,
with an amine of formula (II) 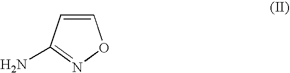
under suitable amide forming conditions, or
(b) reacting a compound of formula (III) 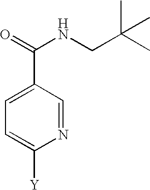
in which Y is halogen,
with a compound of formula (VIIIA) or (VIIIB) 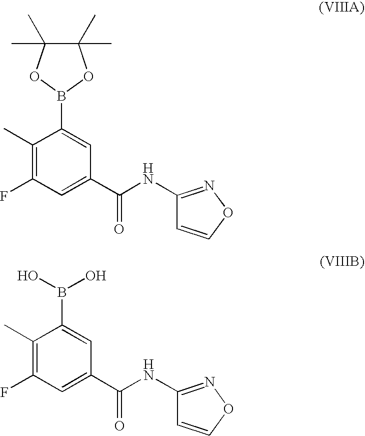
in the presence of a catalyst.

9. A crystalline form of N-(2,2-dimethylpropyl)-6-{3-fluoro-5-[(3-isoxazolylamino)carbonyl]-2-methyl phenyl}-3-pyridinecarboxamide (FORM 1). characterised by substantially the same X-ray powder diffraction (XRPD) pattern as shown in FIG. 1, wherein the XRPD pattern is expressed in terms of 2 theta angles and obtained with a diffractometer using copper Kα-radiation and/or substantially the same differential scanning calorimetry (DSC) thermograms as shown in FIG. 6 wherein the DSC was performed at a scan rate of 10° per minute using a loosely covered aluminium pan.

10. A crystalline form of N-(2,2-dimethylpropyl)-6-{3-fluoro-5-[(3-isoxazolylamino)carbonyl]-2-methylphenyl}-3-pyridinecarboxamide (FORM 2) characterised by substantially the same X-ray powder diffraction (XRPD) pattern as shown in FIG. 2, wherein the XRPD pattern is expressed in terms of 2 theta angles and obtained with a diffractometer using copper Kα-radiation and/or substantially the same differential scanning calorimetry (DSC) thermograms as shown in FIG. 7 wherein the DSC was performed at a scan rate of 10° per minute using a loosely covered aluminium pan.

11. A crystalline form of N-(2,2-dimethylpropyl)-6-{3-fluoro-5-[(3-isoxazolylamino)carbonyl]-2-methyl phenyl}-3-pyridinecarboxamide (FORM 3) characterised by substantially the same X-ray powder diffraction (XRPD) pattern as shown in FIG. 3, wherein the XRPD pattern is expressed in terms of 2 theta angles and obtained with a diffractometer using copper Kα-radiation.

12. A crystalline form of N-(2,2-dimethylpropyl)-6-{3-fluoro-5-[(3-isoxazolylamino)carbonyl]-2-methylphenyl}-3-pyridinecarboxamide (FORM 4) characterised by substantially the same X-ray powder diffraction (XRPD) pattern as shown in FIG. 4, wherein the XRPD pattern is expressed in terms of 2 theta angles and obtained with a diffractometer using copper Kα-radiation.

13. A crystalline form of N-(2,2-dimethylpropyl)-6-{3-fluoro-5-[(3-isoxazolylamino)carbonyl]-2-methyl phenyl}-3-pyridinecarboxamide (FORM 5) characterised by substantially the same X-ray powder diffraction (XRPD) pattern as shown in FIG. 5, wherein the XRPD pattern is expressed in terms of 2 theta angles and obtained with a diffractometer using copper Kα-radiation.

14. A pharmaceutical composition comprising a compound according to claim 2, in association with one or more pharmaceutically acceptable excipients, diluents and/or carriers.
